(19) United States
(12) Patent Application Publication
(10) Pub. No.: US 2023/0200892 A1
(43) Pub. Date: Jun. 29, 2023

(54) STYLET, SYSTEM INCLUDING STYLET, AND METHOD FOR CARRYING OUT MEDICAL PROCEDURE WITH STYLET

(71) Applicant: Boston Scientific Medical Device Limited, Galway (IE)

(72) Inventors: Brock Miller, Toronto (CA); Matthew Gravett, Milton (CA)

(21) Appl. No.: 18/179,670

(22) Filed: Mar. 7, 2023

Related U.S. Application Data
(63) Continuation of application No. PCT/IB2021/057592, filed on Aug. 18, 2021.
(60) Provisional application No. 63/076,458, filed on Sep. 10, 2020.

(51) Int. Cl. A61B 18/14

(52) U.S. Cl. CPC A61B 18/1482; A61B 2018/00601

(57) ABSTRACT

A system of medical devices includes a radiofrequency generator, a stylet, and an introducer. The stylet includes an elongate shaft having a shaft proximal portion and a shaft distal portion defining a shaft distal end. The shaft is rigid. A radiofrequency puncture electrode is at the shaft distal end. The radiofrequency puncture electrode is electrically connectable to the radiofrequency generator. The introducer includes an elongate tube extending between a tube proximal portion and a tube distal portion defining a tube distal end. The shaft is receivable in the tube with the electrode proud of the tube distal end.

### CROSS-REFERENCE TO RELATED APPLICATIONS:

This application is a continuation of and claims the benefit of International Application Number PCT/IB2021/057592, entitled “STYLET, SYSTEM INCLUDING STYLET, AND METHOD FOR CARRYING OUT MEDICAL PROCEDURE WITH STYLET,” and filed Aug. 18, 2021, which claims the benefit of U.S. Provisional Application No. 63/076,458, entitled STYLET, SYSTEM INCLUDING STYLET, AND METHOD FOR CARRYING OUT MEDICAL PROCEDURE WITH STYLET,” and filed Sep. 10, 2020, which are hereby incorporated by reference in their entireties.

### FIELD

This document relates to medical devices. More specifically, this document relates to medical devices such as stylets, systems including such stylets, and methods for using such stylets.

### SUMMARY

The following summary is intended to introduce the reader to various aspects of the detailed description, but not to define or delimit any invention.

Stylets are disclosed. According to some aspects, a stylet includes an elongate shaft having a proximal portion and a distal portion. The shaft is rigid. The distal portion defines a distal end that is blunt. A radiofrequency puncture electrode is at the distal end. The radiofrequency puncture electrode is electrically connectable to a radiofrequency generator.

In some examples, the shaft includes a metallic body and an electrically insulative material on the metallic body. The radiofrequency puncture electrode can be an electrically exposed tip of the metallic body, or a metallic piece joined to the metallic body.

In some examples, the stylet further includes a hub joined to the proximal portion of the shaft. The hub can be configured to electrically connect the metallic body to the radiofrequency generator, for delivery of radiofrequency energy along the metallic body from the proximal portion of the shaft to the radiofrequency puncture electrode.

In some examples, the metallic body includes a lumen extending therethrough from the proximal portion to the distal portion. The distal portion can include one or more ports in fluid communication with the lumen.

In some examples, the stylet further includes a lock mounted to the proximal portion for securing the stylet to an introducer.

Systems of medical devices are also disclosed. According to some aspects, a system of medical devices includes a radiofrequency generator, a stylet, and an introducer. The stylet includes an elongate shaft having a shaft proximal portion and a shaft distal portion defining a shaft distal end. The shaft is rigid. A radiofrequency puncture electrode is at the shaft distal end. The radiofrequency puncture electrode is electrically connectable to the radiofrequency generator. The introducer includes an elongate tube extending between a tube proximal portion and a tube distal portion defining a tube distal end. The shaft is receivable in the tube with the electrode proud of the tube distal end.

In some examples, the shaft distal end is blunt.

In some examples, the stylet is lockable to the introducer with the shaft received in the tube and the electrode proud of the tube distal end.

In some examples, the shaft includes a metallic body and the tube includes an electrically insulative material.

In some examples, the tube proximal portion has a first diameter, and a distal tip of the tube distal portion has second diameter that is less than the first diameter.

Methods for carrying out medical procedures are also disclosed. According to some aspects, a method for carrying out a medical procedure includes: a. with a stylet received in an introducer, percutaneously advancing the stylet and introducer towards a patient's heart; and b. delivering radiofrequency energy from an electrode of the stylet to puncture tissue blocking a path of the stylet and introducer.

In some examples, the tissue is fibrous tissue.

In some examples, the method further includes contacting the heart with the electrode, and delivering radiofrequency energy from the electrode to puncture a pericardium of the heart. The method can further include advancing a distal end of the stylet and a distal end of the introducer through the puncture. The method can further include retracting the stylet from the introducer.

In some examples, the method further includes contacting the heart with the electrode, withdrawing the stylet from the introducer, advancing a secondary radiofrequency puncture device through the introducer towards the heart, and delivering radiofrequency energy from the secondary radiofrequency puncture device to puncture a pericardium of the heart.

In some examples, in step a., the stylet is locked to the introducer.

In some examples, the method includes delivering a contrast agent through the stylet and out of a port of the stylet.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of a system of medical devices, in an assembled state;

FIG. 2 is a perspective view of the system of FIG. 1, in an unassembled state;

FIG. 3 is a cross-section taken along line 3-3 in FIG. 1;

FIG. 4 is a schematic view of a step of a method for carrying out a medical procedure;

FIG. 5 is a schematic view showing a step subsequent to that of FIG. 4;

FIG. 6 is a schematic view showing a step subsequent to that of FIG. 5; and

FIG. 7 is a schematic view showing a step subsequent to that of FIG. 6.

### DETAILED DESCRIPTION:

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are stylets that can be used in various medical procedures, such as pericardial puncture procedures. The stylets are configured to deliver radiofrequency (RF) energy to tissue, in order to puncture the tissue. This can facilitate passage through the body to access a body cavity, as well as a target tissue site. For example, a stylet can be percutaneously advanced towards a target location in a patient's body, such as the heart. If difficulty is encountered in traversing towards the target site due to the presence of impassable tissue (e.g. fibrous tissue) in the pathway of the stylet, RF energy can be delivered from the stylet in order to puncture the tissue. Furthermore, once at the target location, the stylet can optionally be used to puncture the target location by the delivery of RF energy.

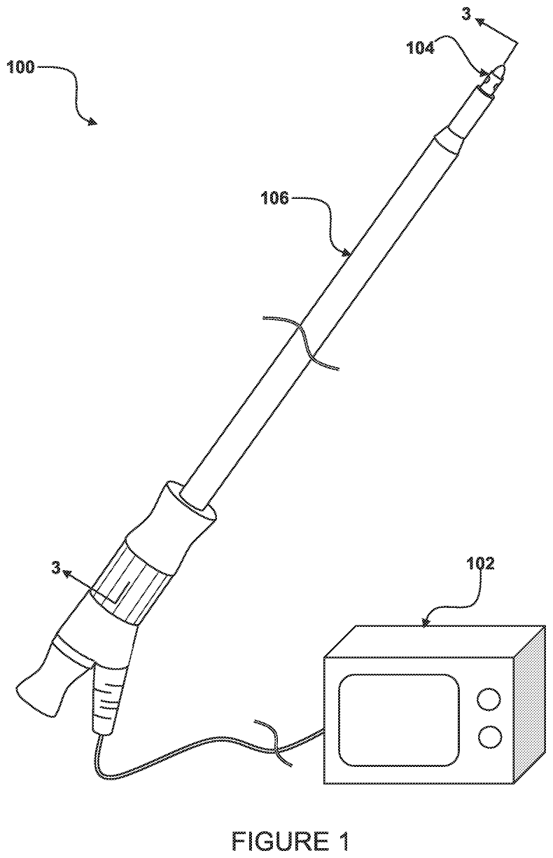

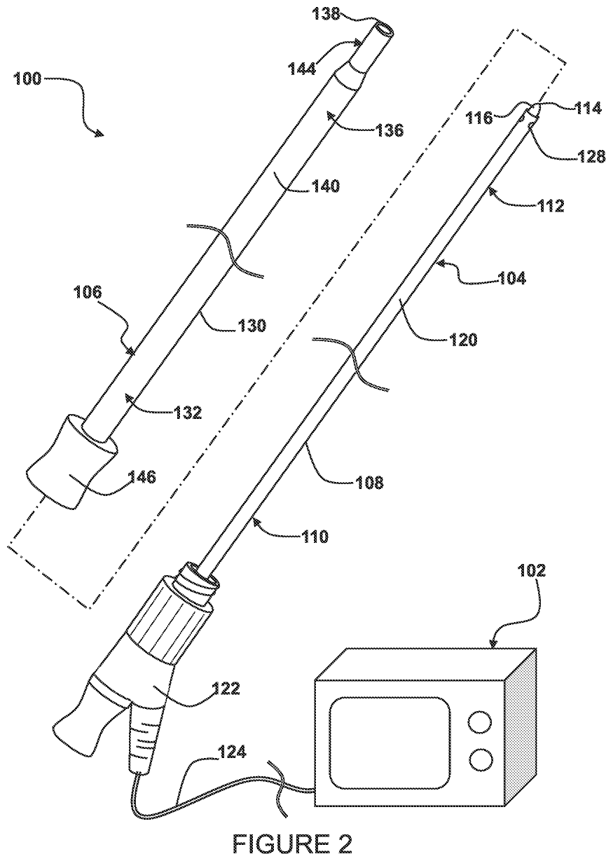

Referring now to FIGS. 1 and 2, a system 100 of medical devices is shown. The system 100 generally includes an RF generator 102, a stylet 104 that is electrically connectable to the RF generator 102, and an introducer 106 in which the stylet 104 is receivable. In FIG. 1, the stylet 104 is received within the introducer 106, and in FIG. 2 the stylet 104 is removed from the introducer 106.

The RF generator 102 can be any suitable RF generator, such as one sold by Baylis Medical Company (Montreal, Canada) under the brand name RFP-100A RF Puncture Generator, and will not be described in detail herein.

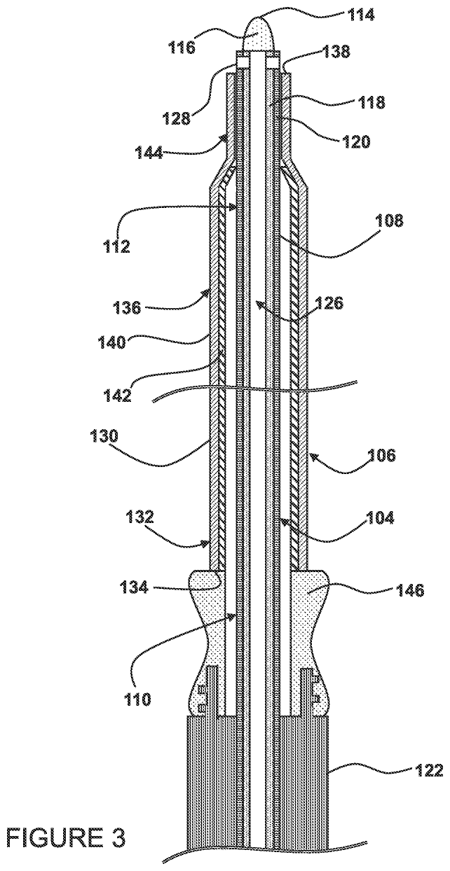

Referring to FIGS. 2 and 3, the stylet 104 generally includes a shaft 108 that is elongate and rigid (i.e. does not significantly bend or flex during normal use). The shaft 108 has a proximal portion 110 (also referred to herein as a ‘shaft proximal portion’) defining a proximal end (not shown, also referred to herein as a ‘shaft proximal end’) and a distal portion 112 (also referred to as a ‘shaft distal portion’) defining a distal end 114 (also referred to herein as a ‘shaft distal end’). The distal end 114 is blunt, in order to avoid or minimize puncturing tissue with only mechanical force.

Referring still to FIGS. 2 and 3, an RF puncture electrode 116 is at the distal end 114 of the shaft 108, and is electrically connectable to the RF generator 102 (not shown in FIG. 3). In the example shown, the shaft 108 includes a metallic body 118 (not visible in FIG. 2) and an electrically insulative material 120 on the metallic body 118, for electrically connecting the electrode 116 to the RF generator 102. For example, the metallic body 118 can be a stainless- steel cannula and the electrically insulative material 120 can be high density polyethylene, and the electrode 116 can be a metallic piece joined to the metallic body (e.g. by soldering or welding). In the example shown, the stylet 104 further includes a hub 122 joined to the proximal portion 110 of the shaft 108, and the hub 122 is configured to electrically connect the metallic body 118 to the RF generator 102. For example, the hub 122 can electrically connect the metallic body to a cable 124 (shown in FIG. 2), which is in turn connectable to the RF generator 102. RF energy can thus be delivered from the RF generator 102 to the cable 124, from the cable 124 to the metallic body 118, and from the metallic body 118 to the electrode 116, which delivers the RF energy to tissue to puncture the tissue.

In alternative examples (not shown), the shaft can include a separate electrical connector for connecting the electrode to the RF generator. For example, a wire can run through the shaft from the electrode, for connection to the RF generator.

In alternative examples (not shown), the electrode can be of another configuration. For example, the electrode can be an electrically exposed tip of the metallic body.

Referring to FIG. 3, in the example shown, the metallic body 118 includes a lumen 126 extending therethrough, from the proximal portion 110 to the distal portion 112. The distal portion 112 further includes fluid ports 128 (only one of which is labelled) in fluid communication with the lumen 126, for delivery of fluid through the lumen 126 from the proximal portion 110 to the distal portion 112 and out of the lumen 126 via the fluid ports 128. As will be described below, the fluid delivered via the fluid ports 128 may be a contrast agent. In the example shown, the distal portion 112 includes two fluid ports. In alternative examples, another number of fluid ports can be provided (i.e. one or more fluid ports).

Referring to FIGS. 2 and 3, the introducer 106 generally includes a tube 130 that is elongate. The tube 130 has a proximal portion 132 (also referred to herein as a ‘tube proximal portion’) defining a proximal end 134 (shown in FIG. 3, also referred to herein as a ‘tube proximal end’) and a distal portion 136 (also referred to as a ‘tube distal portion’) defining a distal end 138 (also referred to herein as a ‘tube distal end’). The distal end 138 of the tube 130 is blunt, in order to avoid or minimize puncturing or damaging tissue.

Referring to FIG. 3, in the example shown, the tube 130 includes an outer layer 140 of a relatively soft polymeric material (e.g. high-density polyethylene), and an inner reinforcing layer 142 (e.g. a stainless steel layer) within the outer layer 140. In the example shown, the inner layer 142 extends to a position shy of the distal end 138 of the tube, leaving a distal tip 144 of the tube 130 without reinforcement. This can minimize the risk of tissue damage due to contact with the introducer 106. Furthermore, the distal tip 144 has a reduced diameter as compared to the proximal portion 132 of the tube 130 (i.e. the proximal portion 132 has a first diameter, and the distal tip 144 has a second diameter that is less than the first diameter). As will be described below, this can facilitate insertion of the distal tip 144 through a puncture. The distal tip 144 can have a length of, for example, between about 1 cm and about 4 cm. In alternative examples, the entire distal tip can be tapered.

Referring still to FIGS. 2 and 3, in the example shown, the introducer 106 further includes a hub 146 joined to the proximal portion 132 of the tube 130.

Optionally, the tube 130 can include a lubricious coating (not shown), to facilitate advancement.

Referring to FIG. 3, the shaft 108 of the stylet 104 is receivable in the tube 130 of the introducer 106. More specifically, as shown in FIG. 3, the shaft 108 of the stylet 104 is receivable in the tube 130 of the introducer 106 with the electrode 116 proud of the tube distal end 138, and with the fluid ports 128 proud of the tube distal end 138. Furthermore, the stylet 104 and introducer 106 are lockable together in the position shown in FIG. 3. More specifically, the hub 122 of the stylet 104 is lockable to the hub 146 of the introducer 106. This locking can be achieved, for example, by a screw luer lock type mechanism.

In the example shown, as described above, the stylet 104 includes an electrically insulative material 120. In alternative examples (not shown), the electrically insulative material of the stylet can be omitted, and the tube of the introducer can serve as an electrically insulative material for the stylet.

In any of the above examples, the stylet and/or introducer can include one or more radiopaque markers, to facilitate visualization under fluoroscopy.

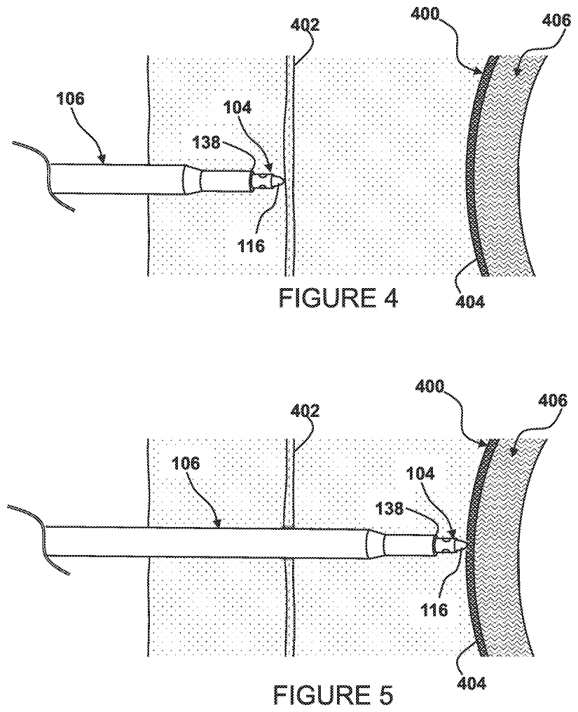

Referring now to FIGS. 4 to 7, a method for carrying out a medical procedure, and more specifically a method for pericardial puncture, will be described. The method will be described with reference to the system 100 of FIGS. 1 to 3; however, the method is not limited to the system 100, and the system 100 is not limited to operation according to the described method.

Referring first to FIG. 4, with the stylet 104 received in and locked to the introducer 106, and with the electrode 116 proud of the tube distal end 138, the stylet 104 and introducer 104 can be percutaneously advanced towards a patient's heart 400. For example, the stylet 104 and introducer 106 can be advanced via the subxiphoid approach. Mechanical force can initially be used to advance the stylet 104 and introducer 106. Referring still to FIG. 4, if tissue 402 (E.g. fibrous tissue) blocks the path of the stylet 104 and introducer 106 so that excessive mechanical force would be required to pass through the tissue 402, RF energy can be delivered from the electrode 116, in order to puncture the tissue 402. This can avoid the use of excessive mechanical force.

Once the tissue 402 has been punctured, delivery of RF energy can be stopped (either automatically at the end of a set pulse, or manually), and the mechanical force can again to be applied to continue to advance the stylet 104 and introducer 106 towards the heart 400. The stylet 104 and introducer 106 can be advanced until the electrode 116 contacts the pericardium 404 of the heart 400, as shown in FIG. 5. Optionally, in order to confirm the position of the stylet 104 and introducer 106, a contrast agent can be delivered through the stylet 104 and out of the fluid ports 128 (not labelled in FIGS. 4 to 7), while viewing the stylet 104 and introducer 106 under fluoroscopy.

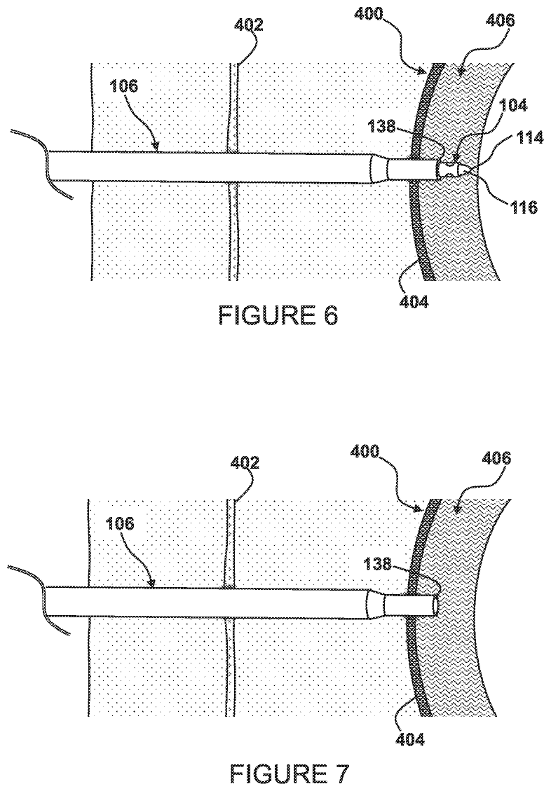

With the position of the stylet 104 and introducer 106 confirmed and with the electrode 116 in contact with the pericardium 404, RF energy can be delivered from the electrode 116 via the RF generator 102 (not shown in FIGS. 4 to 7), to puncture the pericardium 404. The shaft distal end 114 and the tube distal end 138 can then be advanced through the puncture and into the pericardial space 406, as shown in FIG. 6. Optionally, in order to again confirm the position of the stylet 104 and introducer 106, a contrast agent can be delivered through the stylet 104 and out of the fluid ports, while viewing the stylet and introducer under fluoroscopy.

Referring to FIG. 7, the stylet 104 (not visible in FIG. 7) can then be retracted from the introducer 106, leaving the distal end 138 in place in the pericardial space 106. A secondary medical device (e.g. a guidewire, not shown) can then be advanced through the introducer 106, for use in subsequent steps of the medical procedure.

In an alternative example (not shown), rather than using the stylet 104 to puncture the pericardium 404, a secondary RF puncture device (not shown) can be used to puncture the pericardium (i.e. the stylet can be used to facilitate advancement towards the heart, but not to puncture the pericardium). For example, once the stylet and introducer have reached the heart (as shown in FIG. 5) with the electrode in contact with the heart, the stylet can be withdrawn from the introducer. A secondary RF puncture device (e.g. an RF guidewire) can be advanced through the introducer towards the heart, and RF energy can be delivered from the secondary RF puncture device to puncture the pericardium.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A stylet comprising:
an elongate shaft having a proximal portion and a distal portion, wherein the shaft is rigid, and wherein the distal portion defines a distal end that is blunt; and
a radiofrequency puncture electrode at the distal end of the shaft, wherein the radiofrequency puncture electrode is electrically connectable to a radiofrequency generator.

2. The stylet of claim 1, wherein the shaft comprises a metallic body and an electrically insulative material on the metallic body.

3. The stylet of claim 2, wherein the radiofrequency puncture electrode comprises an electrically exposed tip of the metallic body.

4. The stylet of claim 2, wherein the radiofrequency puncture electrode comprises a metallic piece joined to the metallic body.

5. The stylet of claim 2, further comprising a hub joined to the proximal portion of the shaft, wherein the hub is configured to electrically connect the metallic body to the radiofrequency generator, for delivery of radiofrequency energy along the metallic body from the proximal portion of the shaft to the radiofrequency puncture electrode.

6. The stylet of claim 2, wherein the metallic body comprises a lumen extending therethrough from the proximal portion to the distal portion, and the distal portion comprises one or more ports in fluid communication with the lumen.

7. The stylet of claim 1, further comprising a lock mounted to the proximal portion for securing the stylet to an introducer.

8. A system of medical devices, comprising:
a radiofrequency generator;
a stylet comprising (i) an elongate shaft having a shaft proximal portion and a shaft distal portion defining a shaft distal end, wherein the shaft is rigid, and (ii) a radiofrequency puncture electrode at the shaft distal end, wherein the radiofrequency puncture electrode is electrically connectable to the radiofrequency generator; and
an introducer comprising an elongate tube extending between a tube proximal portion and a tube distal portion defining a tube distal end, wherein the shaft is receivable in the tube with the electrode proud of the tube distal end.

9. The system of claim 8, wherein the shaft distal end is blunt.

10. The system of claim 8, wherein the stylet is lockable to the introducer with the shaft received in the tube and the electrode proud of the tube distal end.

11. The system of claim 8, wherein the shaft comprises a metallic body and the tube comprises an electrically insulative material.

12. The system of claim 8, wherein tube proximal portion has a first diameter, and a distal tip of the tube distal portion has second diameter that is less than the first diameter.

13. A method for carrying out a medical procedure:
a. with a stylet received in an introducer, percutaneously advancing the stylet and introducer towards a patient's heart; and
b. delivering radiofrequency energy from an electrode of the stylet to puncture tissue blocking a path of the stylet and introducer.

14. The method of claim 13, wherein the tissue is fibrous tissue.

15. The method of claim 13, further comprising contacting the heart with the electrode, and delivering radiofrequency energy from the electrode to puncture a pericardium of the heart.

16. The method of claim 15, further comprising advancing a distal end of the stylet and a distal end of the introducer through the puncture.

17. The method of claim 16, further comprising retracting the stylet from the introducer.

18. The method of claim 13, further comprising contacting the heart with the electrode, withdrawing the stylet from the introducer, advancing a secondary radiofrequency puncture device through the introducer towards the heart, and delivering radiofrequency energy from the secondary radiofrequency puncture device to puncture a pericardium of the heart.

19. The method of claim 13, wherein in step a., the stylet is locked to the introducer.

20. The method of claim 13, further comprising delivering a contrast agent through the stylet and out of a port of the stylet.
